(19) United States
(12) Patent Application Publication
(10) Pub. No.: US 2025/0161174 A1
(43) Pub. Date: May 22, 2025

(54) STABLE DISPERSIONS OF PROTEIN-ENCAPSULATED PIGMENTS

(71) Applicant: CAPOL INC., St-Hubert (CA)

(72) Inventors: Nsa’nkwe’ni NSISSAE YOH’NSI, Longueil (CA); Pablo ELIZONDO, Montreal (CA)

(21) Appl. No.: 18/953,498

(22) Filed: Nov. 20, 2024

Related U.S. Application Data
(60) Provisional application No. 63/601,578, filed on Nov. 21, 2023.

(51) Int. Cl. A61K 8/11; A23D 9/007; A23P 10/30; A61K 8/64; A61K 8/92; A61Q 1/02

(52) U.S. Cl. CPC A61K 8/11; A23D 9/007; A23P 10/30; A61K 8/64; A61K 8/922; A61Q 1/02; A61K 2800/43

(57) ABSTRACT

Stable dispersions of encapsulated pigments suitable for use in food, cosmetic, pharmaceutical, or personal care products are described herein. Pigments encapsulated in a water-insoluble protein matrix, in particulate form, are homogeneously dispersed in a sol colloid formed by high-shear mixing between a solid fat and a liquid oil. Within the liquid oil, the solid fat's creaming tendency is believed to counteract the sedimentation tendency of the encapsulated pigment, resulting in a dispersions with improved stability.

### RELATED APPLICATION DATA

The present application claims priority to U.S. Provisional Application No. 63/601,578, filed on Nov. 21, 2023, which is incorporated herein by reference in its entirety.

### FIELD

The present description relates to dispersions of protein-encapsulated pigments suitable for use in the food, cosmetic, pharmaceutical, and personal care industries, as alternatives to corresponding powdered pigments. More specifically, the present description relates to protein-encapsulated pigments in particulate form homogeneously dispersed in a sol colloid formed between a solid fat and a liquid oil.

The present description refers to a number of documents, the contents of which are herein incorporated by reference in their entirety.

### BACKGROUND

Pigments for coloring applications generally come in the form of a powder. Alternatives to powdered pigments include liquid or aerosol formulations of pigments. Such formulations present with different problems, almost all related to destabilization events, including phase separation, sedimentation, clarification, creaming, particle aggregation and the like. These destabilization events can lead to uneven application of the pigment, shortened shelf-life of the product, supplemental handling steps for the user before application, such as re-dispersion of the composition. Therefore, there is a need for the development of non-powdered coloring agents formulated in a longer-lasting, stable, and easy to handle composition.

### SUMMARY

In a first aspect, described herein is a pigment dispersion comprising, or consisting essentially of, a sol colloid formed between a solid fat and a liquid oil, and an encapsulated pigment in particulate form homogenously dispersed therein, the pigment being encapsulated in a water-insoluble protein matrix and being present in sufficient quantity to increase the stability of the pigment dispersion as compared to in the absence of the encapsulated pigment.

In a further aspect, described herein is a process for preparing a pigment dispersion, the process comprising mixing a solid fat, a liquid oil, and an encapsulated pigment in particulate form, the pigment being encapsulated in a water-insoluble protein matrix, to form a sol colloid having a stability greater than that of a corresponding sol colloid lacking the encapsulated pigment.

In a further aspect, described herein is a pigment dispersion produced by a process described herein. In a further aspect, described herein is a food, cosmetic, pharmaceutical, or personal care product comprising a pigment dispersion as described herein.

In a further aspect, described herein is a process for increasing the stability of a sol colloid formed between a solid fat and a liquid oil, the process comprising homogenously dispersing therein an extruded water-insoluble protein matrix in particulate form in sufficient quantity to increase the stability of the sol colloid as compared to in the absence of the extruded water-insoluble protein matrix.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the appended drawings:

FIG. 1A to 1D shows the results of turbidimetry measurements using a Turbiscan™ stability analyzer for formulations 3-1 (FIG. 1A), 3-2 (FIG. 1B), 3-3 (FIG. 1C), and 3-4 (FIG. 1D) of Example 3 over the course of 7 days at 25° C. The upper graphs show the change in signal transmission (ΔT %) and the lower graphs show the change in backscattering (ABS %) signal, both as a function of sample height, going from the bottom of the sample (left) to the top of the sample (right). The far-right side of each graph is the correspondence between the color of the curves of the graph and the time in days, hours, and minutes for the 7-day measurements. Each scan is represented by a differently colored curve, with the first curve in blue and the last in red to display exactly when a destabilization is occurring in the analysis. Particular destabilization events, such as clarifications and sedimentations, are indicated with rectangles and arrows.

FIG. 2A to 2D show photographs of formulations 3-1 (FIG. 1A), 3-2 (FIG. 1B), 3-3 (FIG. 1C), and 3-4 (FIG. 1D) of Example 3 taken on day 7 of the stability measurement.

FIG. 3 shows the Turbiscan™ stability index (TSI) of formulations 3-1 (cross), 3-2 (triangle), 3-3 (circle) and 3-4 (square) over time (in days).

FIG. 4A to 4D shows the results of turbidimetry measurements using a Turbiscan™ stability analyzer for formulations 4-1 (FIG. 4A), 4-2 (FIG. 4B), 4-3 (FIG. 4C), and 4-4 (FIG. 4D) of Example 4 over the course of 7 days at 25° C.

FIG. 5A to 5D show photographs of formulations 4-1 (FIG. 5A), 4-2 (FIG. 5B), 4-3 (FIG. 5C), and 4-4 (FIG. 5D) of Example 4, taken on day 7 of the stability measurement.

FIG. 6 shows the Turbiscan™ stability index (TSI) of formulations 4-1 (circle), 4-2 (square), 4-3 (diamond) and 4-4 (triangle) over time (days).

### DETAILED DESCRIPTION

Described herein are homogenous dispersions of encapsulated pigments and processes for their preparation. The encapsulation of pigments, particularly labile and/or water-soluble pigments, in a water-insoluble protein-rich matrix via melt extrusion has been described for example in U.S. Pat. Nos. 9,687,807 and 10,981,136, and in PCT application number PCT/CA2023/050903. While these powdered encapsulated pigments address many industry needs, there are some instances where liquid-based dispersions are preferable for particular applications and/or for their ease of use over powders. However, the tendency of these powdered encapsulated pigments to sediment in the context of a liquid dispersion, thus breaking homogeneity of the dispersion, is a significant drawback. In some aspects, the present subject-matter stems from the demonstration herein that stable dispersions may be prepared by dispersing the protein-encapsulated pigments in a sol colloid formed by high-shear mixing between a solid fat and a liquid oil. Without being bound by theory, the solid fat's creaming tendency is believed to counteract the encapsulated pigment's tendency to sediment, resulting in dispersions with improved stability.

In a first aspect, described herein is a dispersion comprising, or consisting essentially of, a colloidal system (e.g., a sol colloid) formed between a solid fat and a liquid oil, and an encapsulated pigment in particulate form homogenously dispersed therein, the pigment being encapsulated in a water-insoluble protein matrix. As used herein, the expression “consisting essentially of” in the context of pigment dispersions or liquid dispersions described herein refers to the principal ingredients that, by themselves, are sufficient to provide increased stability and homogeneity of the dispersions, but does not exclude the inclusion of other components (e.g., additives, stabilizers, preservatives) in lesser relative quantities so long as the other components do not abrogate the functionality imparted by the principal ingredients.

As used herein, the term “liquid” in the context of liquid dispersions or liquid oils described herein, unless otherwise specified, generally refers the dispersion or oil having a liquid state at the desired usage or manufacturing temperature. In some embodiments, the desired usage or manufacturing temperature may be room temperature (i.e., 20 to 22° C.). In some embodiments, the desired usage or manufacturing temperature may be above room temperature. As used herein, the term “solid” in the context of solid fats or solid forms of pigment dispersions described herein, unless otherwise specified, generally refers the fat or dispersion having a solid state (e.g., paste) at the desired usage or manufacturing temperature. In some embodiments, the desired usage or manufacturing temperature may be room temperature (i.e., 20 to 22° C.). In some embodiments, the desired usage or manufacturing temperature may be above room temperature.

In some embodiments, the solid fat described herein may be present in the pigment dispersion in sufficient quantity to decrease sedimentation of the encapsulated pigment as compared to when the encapsulated pigment is dispersed in the liquid oil alone. In some embodiments, the weight ratio of solid fat to liquid oil in the pigment dispersion described herein may be from about 1:5 to 1:1, 1:4.5 to 1:1.5, 1:4 to 1:2, 1:3.5 to 1:2.5, or 1:3. In some embodiments, the solid fat described herein may be a solid fat that creams within 1, 2, 3, 4, 5, 6, or 7 days after mixing with the liquid oil alone. In some embodiments, the solid fat described herein may be one that has a slip melting point that is at least 5, 6, 7, 8, 9, or 10° C. above room temperature, or above the usage temperature of the pigment dispersion. In some embodiments, the solid fat described herein may be one that has a slip melting point that is at least 27, 28, 29, 30, 31, 32, 33, or 34° C. In some embodiments, the solid fat described herein may comprise or consist of a food-grade solid fat. In some embodiments, the solid fat described herein may be a fat that is white or sufficiently white to avoid negatively affecting the color of the encapsulated pigment. In some embodiments, the solid fat described herein may be a plant fat, an animal fat, a shortening (e.g., vegetable shortening), a butter, a lard, a tallow, a hydrogenated fat, or any combination thereof. In particular embodiments, the solid fat described herein may comprise or consist of palm shortening and/or cocoa butter. In some embodiments, the solid fat described herein may be present in the pigment dispersion at a concentration of about 5% to 30%, 5% to 25%, 10% to 20%, or 15% w/w. In some embodiments, the solid fat described herein may be present in the pigment dispersion at a concentration of about 5, 6, 7, 8, 9, 10, 11, 12, 13, 14, 15, 16, 17, 18, 19, 20, 21, 22, 23, 24, 25, 26, 27, 28, 29, 30% w/w.

In some embodiments, the liquid oil described herein may be present in the pigment dispersion in sufficient quantity such that the dispersion remains in liquid form at a desired usage temperature or the desired manufacturing temperature. As used herein, the expression “desired usage temperature” refers to the temperature at which the pigment dispersion described herein will be applied to a product (e.g., in order to impart or affect the product's color). For greater clarity, the product to which the pigment dispersion is applied may be a product that is an intermediate of a final vendable product. In some embodiments, the desired usage temperature may be room temperature (i.e., 20 to 22° C.), and the liquid oil may be present in the pigment dispersion in sufficient quantity such that the dispersion remains in liquid form at room temperature. In some embodiments, the desired usage temperature may be above room temperature, and the liquid oil may be present in the pigment dispersion in sufficient quantity such that the dispersion remains in liquid form at the desired usage temperature (which is above room temperature). In such embodiments, the pigment dispersions described herein may have a solid state (e.g., a paste) at room temperature, and then may be warmed at the moment of use to be transformed into a liquid state. While such an embodiment may involve an additional warming step (e.g., during manufacturing and/or at the moment of usage), adopting a solid or paste form may be beneficial for longer-term stability.

As used herein, the expression “desired manufacturing temperature” refers to the temperature at which the pigment dispersion described herein is produced (e.g., in a process described herein). In some embodiments, the desired manufacturing temperature may be room temperature so as to avoid an unnecessary active warming step (e.g., during high-shear mixing of ingredients). In some embodiments, the desired manufacturing temperature may be above room temperature but below the slip melting point of the solid fat (e.g., so as not to interfere with sol colloid formation), which may require active and/or passive warming (e.g., during high-shear mixing of ingredients). Such embodiments, may be pertinent for example for pigment dispersions that are to be at solid state at room temperature and that may require a warming step at the moment of usage to be transformed back into liquid form.

In some embodiments, the liquid oil described herein may comprise a food-grade oil. In some embodiments, the liquid oil described herein may comprise a plant oil (e.g., a vegetable oil), an animal oil, a mineral oil (e.g., paraffin oil), an oil blend, or any combination thereof. In some embodiments, the liquid oil described herein may comprise or consist of canola oil, medium chain triglyceride (MCT) oil, avocado oil, corn oil, cottonseed oil, flaxseed oil, nut (almond, beech, Brazil nut, cashew, Jamaican cobnut, hazelnut, macadamia, mongongo, pecan, pine nut, pistachio, walnut, peanut, pumpkin seed) oils, olive oil, grape seed oil, palm oil, palm kernel oil, rapeseed oil, safflower oil, sesame oil, soybean oil, sunflower oil, theobroma oil, oils obtained from fruits such as citrus, including grapefruit, lemon, orange, oils derived or obtained from melon and gourd seeds, oils derived or obtained from spices, including coriander, oils obtained from cereal, including quinoa, rice, rice bran or wheat germ. In some embodiments, the liquid oil described herein may be present in the pigment dispersion at a concentration of about 25% to 65%, 30% to 60%, 35% to 55%, 40% to 50%, or 45% w/w. In some embodiments, the liquid oil described herein may be present in the pigment dispersion at a concentration of about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65% w/w.

In some embodiments, the encapsulated pigment described herein may be encapsulated in a water-insoluble protein matrix via extrusion encapsulation, for example as described in U.S. Pat. Nos. 9,687,807 and 10,981,136, and in PCT application number PCT/CA2023/050903. In some embodiments, the protein matrix described herein may comprise or consist of extruded plant protein, extruded animal protein, or a mixture thereof. In some embodiments, the protein matrix described herein may comprise or consist of extruded rice protein, extruded pea protein, extruded soy protein, extruded whey protein, extruded sunflower protein, extruded oat protein, extruded fibrous plant protein, or any combination thereof. In some embodiments, the pigment dispersion described herein may comprise or consist of at least two encapsulated pigments of different hues and/or opacities, enabling a broad spectrum of potential colors. In some embodiments, the pigment encapsulated may be a natural dye.

In some embodiments, the encapsulated pigment may be present in a pigment dispersion described herein in sufficient quantity to increase the stability of the pigment dispersion as compared to in the absence of the encapsulated pigment. In some embodiments, the increase in the stability of the pigment dispersion may comprise decreased sedimentation, decreased creaming, decreased clarification, decreased phase separation, or any combination thereof. Such instability may be monitored, for example, by visual inspection over a given period of time (e.g., days, weeks, or months), or via analytical methods such as by turbidimetry. In some embodiments, the encapsulated pigment present in the pigment dispersion described herein may be between 10% to 60%, 10% to 55%, 15% to 55%, 20% to 55%, 20% to 50%, 25% to 50%, 30% to 50%, 30% to 45%, 35% to 45%, or about 40% w/w. In some embodiments, the encapsulated pigment may be present in the pigment dispersion described herein at a concentration of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60% w/w.

In a further aspect, described herein is a process for preparing a pigment dispersion. The process generally comprises mixing a solid fat, a liquid oil, and an encapsulated pigment in particulate form, the pigment being encapsulated in a water-insoluble protein matrix, to form a sol colloid. In embodiments, the pigment dispersion comprising the encapsulated pigment has a stability greater than that of a corresponding sol colloid lacking the encapsulated pigment. In some embodiments, at least the solid fat and the liquid oil are mixed under conditions of hydraulic shear sufficiently high to produce a sol colloid, wherein the temperature during sol colloid formation does not exceed the slip melting temperature of the solid fat. In some embodiments, the solid fat and the liquid oil are mixed under conditions of hydraulic shear sufficiently high to produce a sol colloid, into which the encapsulated pigment in particulate form is then dispersed under conditions of lower hydraulic shear (e.g., low or medium hydraulic shear). In some embodiments, at least the solid fat and the liquid oil may be advantageously mixed using a colloid mill.

In a further aspect, described herein is a pigment dispersion produced by a process described herein. In a further aspect, the pigment dispersion described herein may be for use in the manufacture of a food, cosmetic, pharmaceutical, or personal care product. In a further aspect, described herein is a food, cosmetic, pharmaceutical, or personal care product comprising the pigment dispersion as described herein.

In a further aspect, described herein is a process for increasing the stability of a sol colloid formed between a solid fat and a liquid oil, the process comprising homogenously dispersing therein an extruded water-insoluble protein matrix in particulate form in sufficient quantity to increase the stability of the sol colloid as compared to in the absence of the extruded water-insoluble protein matrix. In some embodiments, the extruded water-insoluble protein matrix may encapsulate an active agent (e.g., a labile active agent). In some embodiments, the active agent may be a pigment described herein and the solid fat, the liquid oil, the encapsulated pigment, the pigment dispersion, or any combination thereof, may be as described herein.

### Items

In some aspects, described herein is one or more of the following items:
1. A pigment dispersion comprising, or consisting essentially of, a sol colloid formed between a solid fat and a liquid oil, and an encapsulated pigment in particulate form homogenously dispersed therein, the pigment being encapsulated in a water-insoluble protein matrix and being present in sufficient quantity to increase the stability of the pigment dispersion as compared to in the absence of the encapsulated pigment.
2. The pigment dispersion of item 1, wherein the solid fat is present in the pigment dispersion in sufficient quantity to decrease sedimentation of the encapsulated pigment as compared to when the encapsulated pigment is dispersed in the liquid oil alone.
3. The pigment dispersion of item 1, wherein the weight ratio of solid fat to liquid oil in the pigment dispersion is from about 1:5 to 1:1, 1:4.5 to 1:1.5, 1:4 to 1:2, 1:3.5 to 1:2.5, or 1:3.
4. The pigment dispersion of any one of items 1 to 3, wherein the solid fat is one that creams (e.g., within 1, 2, 3, 4, 5, 6, or 7 days) following mixing with the liquid oil in the absence of the encapsulated pigment.
5. The pigment dispersion of any one of items 1 to 4, wherein the slip melting point of the solid fat is above 27, 28, 29, 30, 31, 32, 33, or 34° C.
6. The pigment dispersion of any one of items 1 to 5, wherein the solid fat comprises or consists of a food-grade solid fat, a white fat, a plant fat, an animal fat, a shortening, a butter, a lard, a tallow, a hydrogenated fat, or any combination thereof.
7. The pigment dispersion of any one of items 1 to 6, wherein the solid fat comprises or consists of palm shortening and/or cocoa butter.
8. The pigment dispersion of any one of items 1 to 7, wherein the solid fat present in the pigment dispersion is about 5% to 30%, 5% to 25%, 10% to 20%, or 15% w/w.
9. The pigment dispersion of any one of items 1 to 8, wherein the liquid oil is present in the pigment dispersion in sufficient quantity such that the dispersion remains in liquid form at a desired usage temperature.
10. The pigment dispersion of any one of items 1 to 9, wherein the liquid oil comprises a food-grade oil, a plant oil (e.g., a vegetable oil), an animal oil, a mineral oil (e.g., paraffin oil), an oil blend, or any combination thereof.
11. The pigment dispersion of any one of items 1 to 10, wherein the liquid oil comprises canola oil and/or medium chain triglyceride (MCT) oil.
12. The pigment dispersion of any one of items 1 to 11, wherein the liquid oil present in the pigment dispersion is about 25% to 65%, 30% to 60%, 35% to 55%, 40% to 50%, or 45% w/w.
13. The pigment dispersion of any one of items 1 to 12, wherein the encapsulated pigment is encapsulated in a water-insoluble protein matrix via extrusion encapsulation.
14. The pigment dispersion of any one of items 1 to 13, wherein the protein matrix comprises or consists of extruded plant protein, extruded animal protein, or a mixture thereof.
15. The pigment dispersion of any one of items 1 to 14, wherein the protein matrix comprises or consists of extruded rice protein, extruded pea protein, extruded soy protein, extruded whey protein, extruded sunflower protein, extruded oat protein, extruded fibrous plant protein, or any combination thereof.
16. The pigment dispersion of any one of items 1 to 15, wherein the pigment dispersion comprises or consists of at least two encapsulated pigments of different hues and/or opacities.
17. The pigment dispersion of any one of items 1 to 16, wherein the encapsulated pigment present in the pigment dispersion is between 10% to 60%, 10% to 55%, 15% to 55%, 20% to 55%, 20% to 50%, 25% to 50%, 30% to 50%, 30% to 45%, 35% to 45%, or about 40% w/w; or wherein the encapsulated pigment present in the pigment dispersion is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60% w/w.
18. The pigment dispersion of any one of items 1 to 17, wherein said increase in the stability of the pigment dispersion comprises decreased sedimentation, decreased creaming, decreased clarification, or any combination thereof.
19. A process for preparing a pigment dispersion, the process comprising mixing a solid fat, a liquid oil, and an encapsulated pigment in particulate form, the pigment being encapsulated in a water-insoluble protein matrix, to form a sol colloid having a stability greater than that of a corresponding sol colloid lacking the encapsulated pigment.
20. The process of item 19, wherein at least the solid fat and the liquid oil are mixed under conditions of hydraulic shear sufficiently high to produce a sol colloid, wherein the temperature during sol colloid formation does not exceed the slip melting temperature of the solid fat.
21. The process of item 19 or 20, wherein the solid fat and the liquid oil are mixed under conditions of hydraulic shear sufficiently high to produce a sol colloid, into which the encapsulated pigment in particulate form is then dispersed under conditions of lower hydraulic shear (e.g., low or medium hydraulic shear).
22. The process of any one of items 19 to 21, wherein at least the solid fat and the liquid oil are mixed using a colloid mill.
23. The process of any one of items 19 to 22, wherein: (a) the solid fat is as defined in any one of items 2 to 8; (b) the liquid oil is as defined in any one of items 9 to 12; (c) the encapsulated pigment is as defined in any one of items 13 to 18; (d) the pigment dispersion is as defined in any one of items 1 to 18; or (e) any combination of (a) to (d).
24. A pigment dispersion produced by the process of any one of items 19 to 23.
25. The pigment dispersion of any one of items 1 to 18, or produced by the process of any one of items 19 to 23, for use in the manufacture of a food, cosmetic, pharmaceutical, or personal care product.
26. A food, cosmetic, pharmaceutical, or personal care product comprising the pigment dispersion of any one of items 1 to 18, or produced by the process of any one of items 19 to 23.
27. A process for increasing the stability of a sol colloid formed between a solid fat and a liquid oil, the process comprising homogenously dispersing therein an extruded water-insoluble protein matrix in particulate form in sufficient quantity to increase the stability of the sol colloid as compared to in the absence of the extruded water-insoluble protein matrix.
28. The process of item 27, wherein the extruded water-insoluble protein matrix encapsulates an active agent (e.g., a labile active agent).
29. The process of item 27 or 28, wherein the active agent is a pigment and wherein: (a) the solid fat is as defined in any one of items 2 to 8; (b) the liquid oil is as defined in any one of items 9 to 12; (c) the encapsulated pigment is as defined in any one of items 13 to 18; (d) the pigment dispersion is as defined in any one of items 1 to 18; or (e) any combination of (a) to (d).

### EXAMPLES

### Example 1: Materials and Methods

### 1.1 Preparation of Water-Insoluble Protein-Encapsulated Pigment Powders

Powdered water-insoluble protein-encapsulated pigments (hereinafter “encapsulated pigments”) described herein were generally prepared as described in U.S. Pat. Nos. 9,687,807 and 10,981,136, or in PCT application number PCT/CA2023/050903, with minor modifications. The encapsulated pigment employed in the results shown in Examples 3 and 4 was curcumin (yellow).

### 1.2 Preparation of the Oil-Based Encapsulated Pigment Dispersions

Fat and oil were premixed together at the desired ratios using a mixer or a kettle paddle to break the fat solids slightly before subjecting the pre-mixture to high-shear mixing conditions using either a lab-scale high-shear mixer or a production-scale colloid mill, to enable the formation of a sol colloid. When the fat and oil were mixed using the lab-scale high-shear mixer, the high-shear mixer was operated at 7000 rpm for 5 minutes. When the fat and oil were mixed using a colloid mill, the colloid mill was operated at 3000 rpm speed, with a gap setting between the rotor and stator of 4 to 5 cm passing the whole volume twice through the colloid mill, at a rate of approximately 1000 kg/hour. Unless otherwise indicated, powdered encapsulated pigments were then dispersed in the colloid by manual stirring or using a motorized mixer operated at a slow-medium speed (e.g., less than 1000 rpm), to obtain an oil-based encapsulated pigment dispersion.

### 1.3 Stability Testing

Stability of the oil-based encapsulated pigment dispersions were assessed qualitatively or semi-quantitatively by visual inspection of the sample over time, or quantitively by turbidimetry using a Turbiscan™ analyzer. For turbidimetry, 20 mL of each oil-based encapsulated pigment dispersion was transferred into 30-mL glass vials and then scanned every hour for at least seven days at 25° C. Each vial was scanned vertically, with data acquired every 20 μm. A near-infrared (NIR) light signal of 880 nm was emitted by the light source of the scanning reading head of the analyzer towards the sample and this signal was subsequently acquired by a transmission detector and a backscattering detector, also located on the scanning reading head. The analyzer scanned the entire sample for each measurement and detected differences in light scattering intensity at all points in the vial. Data corresponding to light transmission levels and backscattering levels over time were recorded. Measurement of the changes (delta (A) change) of these parameters over time provides information about particle migration (sedimentation, clarification and creaming) in the sample and particle size change (flocculation and coalescence), which are indicative of macroscopic stability or instability of the sample. Turbiscan Stability Index (TSI) is an automatic calculation that sums all destabilization data (transmission and backscattering variations) into a single number reflective of the sample's overall stability. The higher the TSI value, the lower the sample's stability.

### Example 2: Enhanced Stability of Encapsulated Pigments Dispersed in Liquid Oil/Solid Fat Colloids

Some of the technical challenges of working with labile and/or water-soluble pigments have been addressed by encapsulating them in a water-insoluble protein-rich matrix via melt extrusion, such as described in U.S. Pat. Nos. 9,687,807 and 10,981,136, and in PCT application number PCT/CA2023/050903. While these powdered encapsulated pigments addressed many industry needs, there are some instances where liquid-based dispersions may be preferred for particular applications and/or for their ease of use over powders. Thus, we set out to develop stable liquid-based dispersions of encapsulated pigments suitable for food, cosmetic, pharmaceutical, and personal-care applications.

Preliminary experiments dispersing the encapsulated pigment powders in a variety of oils or oil-blends resulted in their rapid undesirable sedimentation. Initial attempts at combatting this sedimentation by adding thickeners (e.g., gums such as xanthan gum or Arabic gum), viscosity-increasing agents (e.g., propylene glycol), or via emulsifications were unsuccessful and resulted in unstable oil-based dispersions. Empirical screening of a variety of food-grade ingredients revealed that the sedimentation could be counteracted when the encapsulated pigments were dispersed in a sol colloid mixture of a liquid oil and a solid fat. It was observed that, in the absence of the encapsulated pigment, sol colloid mixtures of the liquid oil and solid fat were unstable, with the fat rising to the surface (i.e., “creaming”) over time. Interestingly, dispersion of the encapsulated pigment powder in the liquid oil/solid fat mixture increased the stability of the overall mixture, resulting in the delay or, in some cases, avoidance of both sedimentation and creaming. These results suggested that the water-insoluble protein-rich matrix of the encapsulated pigment particles were actively participating, along with the liquid oil/solid fat sol colloid mixture, in the overall stability of the oil-based encapsulated pigment dispersion.

Solid fat to liquid oil weight ratios of about 1:5 to about 1:1, with encapsulated pigments ranging from 10% to 60% w/w (based on the final dispersion), were associated with oil-based encapsulated pigment dispersions having increased stability as evaluated by visual inspection, as compared to corresponding formulations lacking the solid fat, provided that the solid fat remained in solid form during high-shear mixing with the liquid oil. This could be ensured by selecting a solid fat having a slip melting point above the maximum temperature reached during high-shear mixing of the solid fat and liquid oil for sol colloid formation.

More thorough quantitative stability testing of informative oil-based encapsulated pigment dispersions was performed in Examples 3 and 4. To reduce the total number of samples to be tested, the weight ratio of solid fat to liquid oil was set to 1:3 for all dispersions. Furthermore, one of the most stabilizing solid fats (palm shortening) and one of the least stabilizing solid fats (coconut oil) were selected for contrasting purposes. Canola oil and MCT oil were selected as examples of suitable liquid oils.

### Example 3: Palm Shortening-Containing Formulations Exhibit Enhanced Stability Over Coconut Oil-Containing Formulations

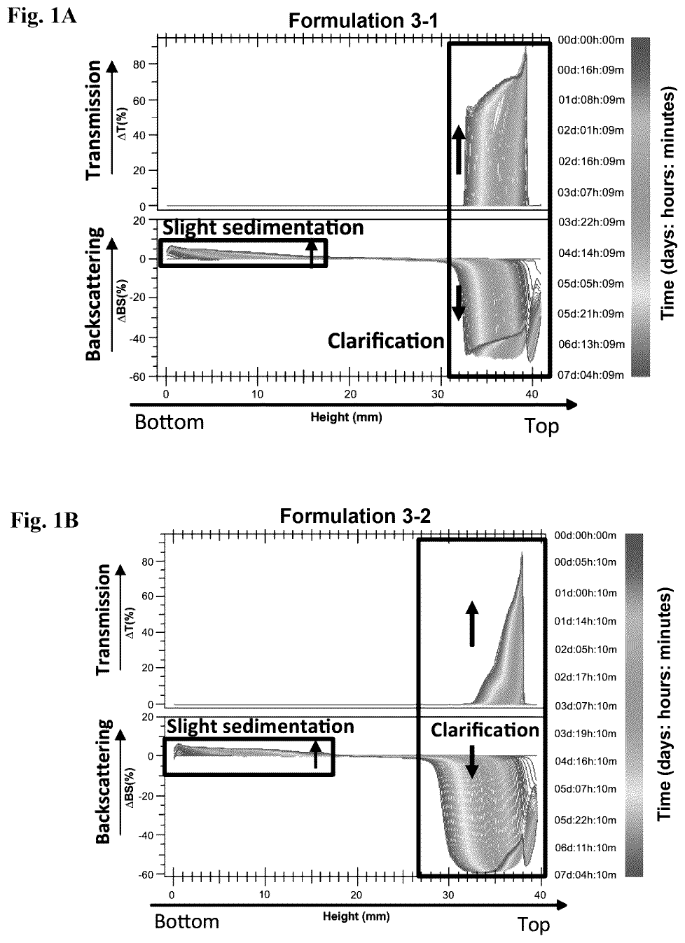

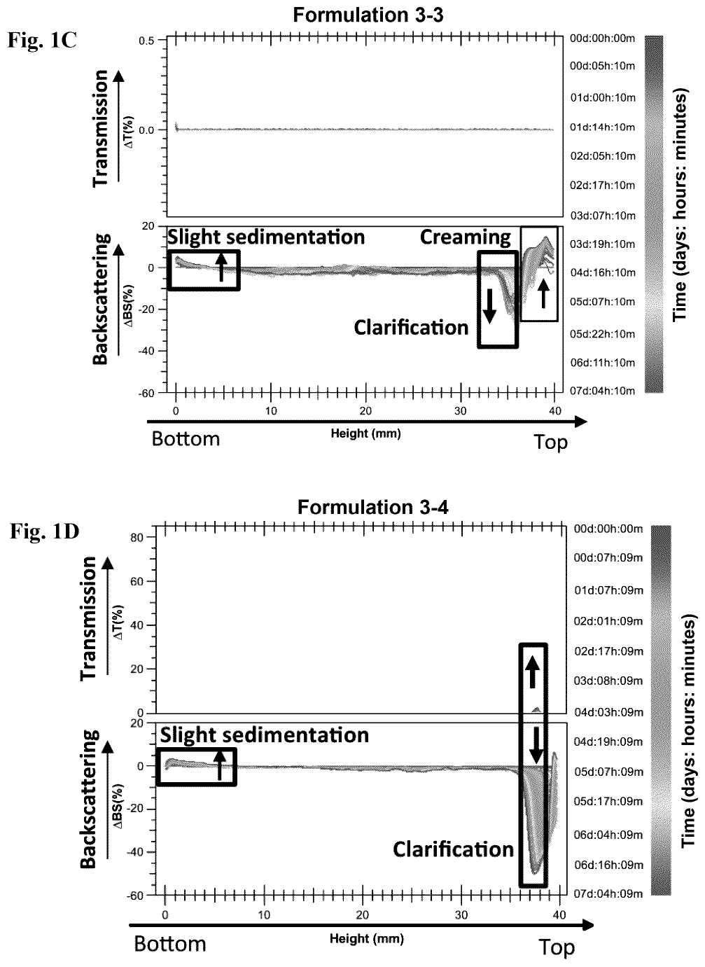

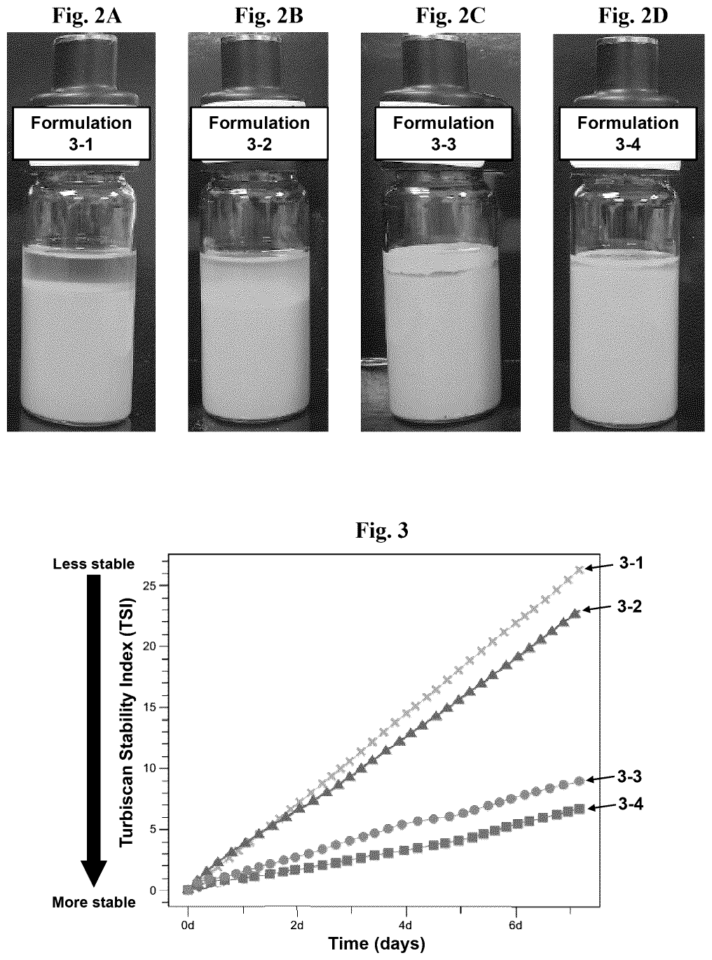

Formulations 3-1 to 3-4 shown in Table 1 were prepared and subjected to quantitative stability testing by turbidimetry as described in Example 1. Briefly, the solid fats and liquid oils were premixed at a 1:3 weight ratio before being subjected to mixing with a lab-scale high-shear mixer to facilitate colloid formation. Encapsulated pigment powders were then immediately dispersed in the solid fat/liquid oil colloid by manual stirring or using a motorized mixer operated at a slow-medium speed. Stability measurements from turbidimetry testing are shown in FIG. 1A-1D. Corresponding images of each sample after 7 days are shown in FIG. 2A-2D. Turbiscan Stability Indexes (TSIs) for each formulation at days 3 and 7 are shown in Table 1, and overall TSI kinetics over the course of the seven days is shown in FIG. 3.

TABLE 1
Composition and comparative stability testing of Formulations 3-1 to 3-4

| | Formulation: | | | |
|---|---|---|---|---|
| | 3-1 | 3-2 | 3-3 | 3-4 |
| Solid fat | 15% coconut oil | 15% coconut oil | 15% palm shortening | 15% palm shortening |
| Liquid oil | 45% canola oil | 45% MCT oil | 45% MCT oil | 45% canola oil |
| Encapsulated pigment | 40% | 40% | 40% | 40% |

Stability:

| | | | | |
|---|---|---|---|---|
| Turbiscan plots | FIG. 1A | FIG. 1B | FIG. 1C | FIG. 1D |
| Image at day 7 | FIG. 2A | FIG. 2B | FIG. 2C | FIG. 2D |
| TSI at day 3 | 10.72 | 9.93 | 4.11 | 2.49 |
| TSI at day 7 | 26.25 | 22.89 | 8.92 | 6.66 |
| Overall TSI kinetics | FIG. 3 | FIG. 3 | FIG. 3 | FIG. 3 |

The results shown in FIG. 1A-1D and FIG. 2A-2D, as well as the TSI values shown in Table 1 and FIG. 3 all demonstrate the increased stability of the palm shortening-containing formulations 3-3 and 3-4, as compared to the coconut oil-containing formulations 3-1 and 3-2. These results illustrate the impact of the type of solid fat on overall dispersion stability, as discussed in Example 2. While coconut oil is a solid fat at room temperature, it has a melting point of about 25° C. To ensure proper colloid formation between the solid fat and the liquid oil, the solid fat should remain in solid form during high-shear mixing with the liquid oil. Although mixing between the solid fat and the liquid oil was performed at room temperature, the temperature of the mixture was observed to rise by several degrees during the mixing process with the lab-scale high-shear mixer. Without being bound by theory, the maximum temperature attained by the mixture during the high-shear mixing may have reached or surpassed the melting point of the coconut oil, thereby interfering with its ability to form a proper sol colloid with the liquid oil. In contrast, the slip melting point of palm shortening is reported to be about 36-38° C., which was well above the maximum temperature reached by the mixture during the high-shear mixing. Thus, the palm shortening is expected to remain in solid form during the high-shear mixing process.

### Example 4: Differential Mixing Speeds of Solid Fat/Liquid Oil Vs. Encapsulated Pigment is Associated with Further Enhanced Stability

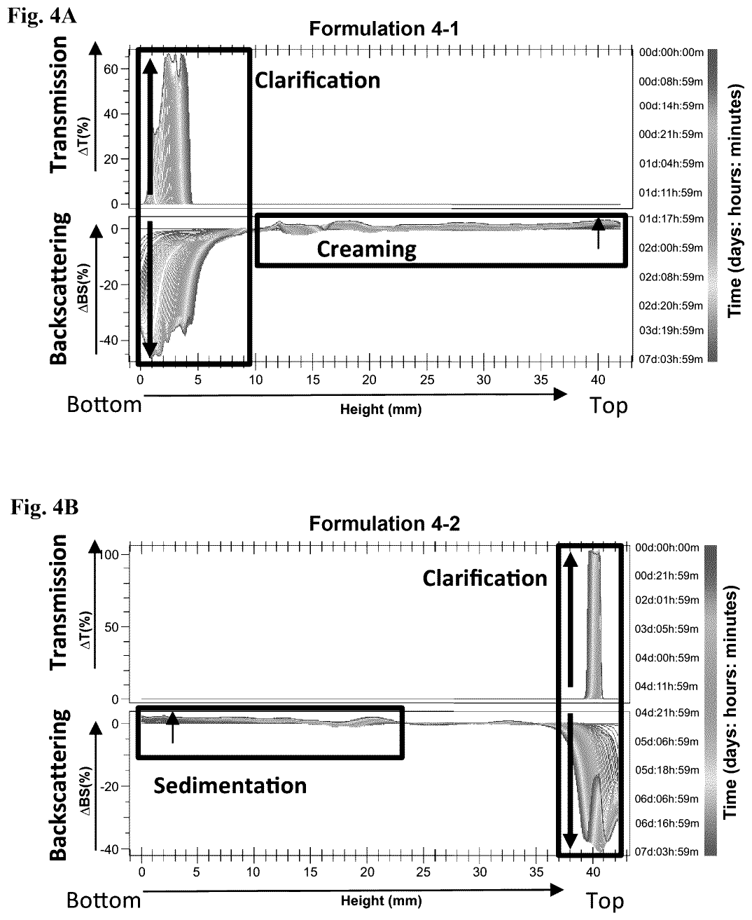

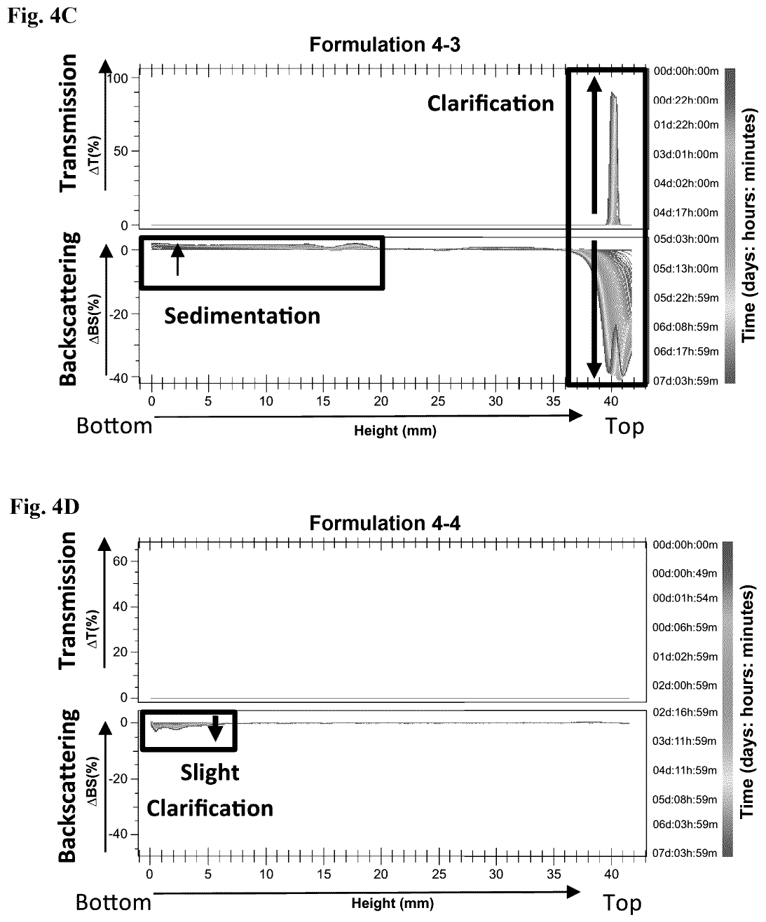

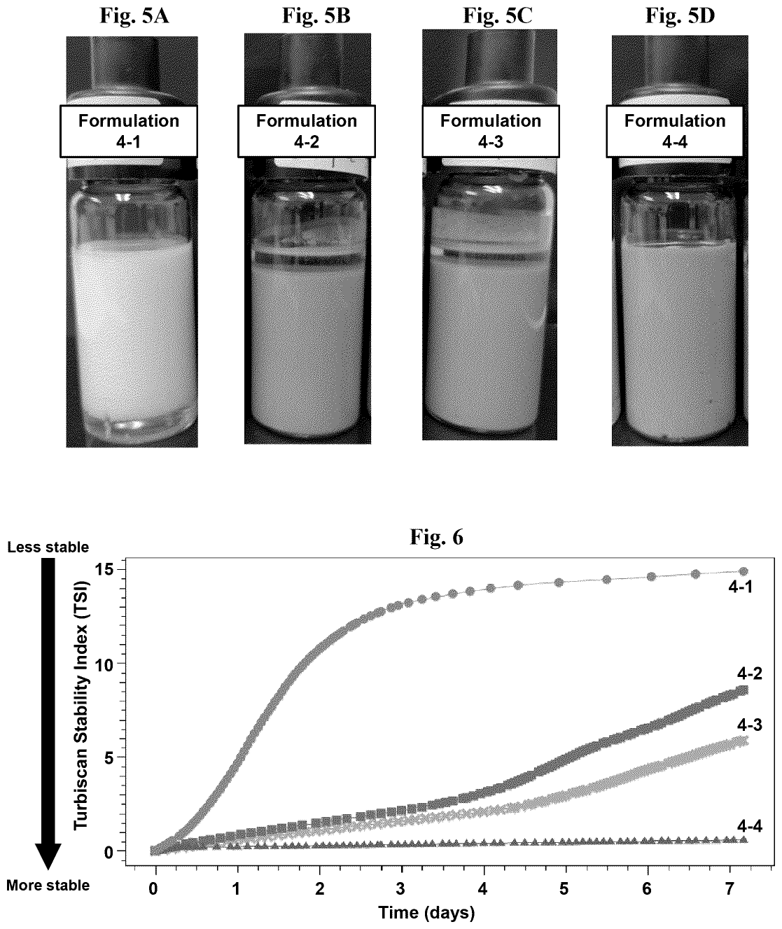

Amongst the formulations in Example 3 prepared using a lab-scale high-shear mixer, formulation 3-4 (15% palm shortening/45% canola oil/40% encapsulated pigment) exhibited the highest stability. To assess whether this result could be replicated using a production-scale colloid mill, corresponding formulations 4-1 to 4-4 shown in Table 2 were prepared by premixing palm shortening with canola oil at a weight ratio of 1:3, and then subjecting the premixture to colloid milling to achieve sol colloid formation. Formulation 4-1 corresponds to the palm shortening/canola oil sol colloid mixture alone before the addition of encapsulated pigment. For formulations 4-2 and 4-3, encapsulated pigment was mixed with the palm shortening/canola oil using the colloid mill—either by including all three ingredients (i.e., palm shortening, canola oil, and encapsulated pigment) in a single colloid milling step, or by adding the encapsulated pigment to freshly prepared formulation 4-1 followed by a second colloid milling step to disperse the encapsulated pigment under high-shearing conditions. Formulation 4-4 was prepared by dispersing the encapsulated pigment in freshly-prepared formulation 4-1 with low shear mixing. Stability measurements from turbidimetry testing are shown in FIG. 4A-4D. Corresponding images of each sample after 7 days are shown in FIG. 5A-5D. Turbiscan Stability Indexes (TSIs) for each formulation at days 3 and 7 are shown in Table 2, and overall TSI kinetics is shown in FIG. 6.

TABLE 2
Composition and comparative stability
testing of Formulations 4-1 to 4-4

| | Formulation: | | | |
|---|---|---|---|---|
| | 4-1 | 4-2 | 4-3 | 4-4 |
| Palm shortening | 25% | 15% | 15% | 15% |
| Canola oil | 75% | 45% | 45% | 45% |
| Encapsulated pigment | — | 40% | 40% | 40% |

Stability:

| | | | | |
|---|---|---|---|---|
| Turbiscan plots | FIG. 4A | FIG. 4B | FIG. 4C | FIG. 4D |
| Image at day 7 | FIG. 5A | FIG. 5B | FIG. 5C | FIG. 5D |
| TSI at day 3 | 13.2 | 2.1 | 1.5 | 0.33 |
| TSI at day 7 | 14.8 | 8.3 | 5.7 | 0.60 |
| Overall TSI kinetics | FIG. 6 | FIG. 6 | FIG. 6 | FIG. 6 |

A colloid mill applies a level of hydraulic shear stress on a fluid much greater than that obtainable using a lab-scale higher-shear mixer, such as the one employed in Examples 2 and 3 to produce the oil-based encapsulated pigment dispersions. Thus, the use of a colloid mill in Example 4 could be expected to produce a stable sol colloid formed by high-shear mixing of the palm shortening and the canola oil. However, this is not what was observed. Formulation 4-1, which corresponds to the colloid milled palm shortening and canola oil, had extremely poor stability kinetics, with significant creaming and clarification being observed after only one day, and the formulation reaching a TSI of 10.7 after only two days (FIG. 4A and FIG. 6). As previously observed by visual inspection with a variety of fat/oil mixtures in Example 2, dispersing the encapsulated pigment in the palm shortening/canola oil sol colloid (formulation 4-1) using either a colloid mill (formulations 4-2 and 4-3) or via low shear mixing (formulation 4-4) significantly stabilized the sol colloid of formulation 4-1 (see FIGS. 4B-4D and FIG. 6). These results demonstrate that the water-insoluble protein-rich matrix of the encapsulated pigment particles indeed play an active role in stabilizing the liquid oil/solid fat sol colloid mixture, as initially observed in Example 2.

Interestingly, formulation 4-4 exhibited a striking level of stability with nearly no signs of instability by turbidimetry testing over seven days (FIG. 4D and FIG. 6). Furthermore, no significant signs of instability were observed upon visual inspection several months after formulation (data not shown). Since it is the only difference between formulation 4-4 and formulations 4-2/4-3, these results suggest that a gentler dispersing (e.g., non-high-shear mixing) of the encapsulated pigment in the liquid oil/solid fat sol colloid mixture favorably impacts stability.

### REFERENCES

U.S. Pat. No. 9,687,807
U.S. Pat. No. 10,981,136
PCT application number PCT/CA2023/050903

1. A pigment dispersion comprising, or consisting essentially of, a sol colloid formed between a solid fat and a liquid oil, and an encapsulated pigment in particulate form homogenously dispersed therein, the pigment being encapsulated in a water-insoluble protein matrix and being present in sufficient quantity to increase the stability of the pigment dispersion as compared to in the absence of the encapsulated pigment.

2. The pigment dispersion of claim 1, wherein the solid fat is present in the pigment dispersion in sufficient quantity to decrease sedimentation of the encapsulated pigment as compared to when the encapsulated pigment is dispersed in the liquid oil alone.

3. The pigment dispersion of claim 1, wherein the weight ratio of solid fat to liquid oil in the pigment dispersion is from about 1:5 to 1:1, 1:4.5 to 1:1.5, 1:4 to 1:2, 1:3.5 to 1:2.5, or 1:3.

4. The pigment dispersion of claim 1, wherein the solid fat is one that creams following mixing with the liquid oil in the absence of the encapsulated pigment.

5. The pigment dispersion of claim 1, wherein the slip melting point of the solid fat is above 27, 28, 29, 30, 31, 32, 33, or 34° C.

6. The pigment dispersion of claim 1, wherein the solid fat comprises or consists of a food-grade solid fat, a white fat, a plant fat, an animal fat, a shortening, a butter, a lard, a tallow, a hydrogenated fat, or any combination thereof.

7. The pigment dispersion of claim 1, wherein the solid fat comprises or consists of palm shortening and/or cocoa butter.

8. The pigment dispersion of claim 1, wherein the solid fat present in the pigment dispersion is about 5% to 30%, 5% to 25%, 10% to 20%, or 15% w/w.

9. The pigment dispersion of claim 1, wherein the liquid oil is present in the pigment dispersion in sufficient quantity such that the dispersion remains in liquid form at a desired usage temperature.

10. The pigment dispersion of claim 1, wherein the liquid oil comprises a food-grade oil, a plant oil, an animal oil, a mineral oil, an oil blend, or any combination thereof.

11. The pigment dispersion of claim 1, wherein the liquid oil comprises canola oil and/or medium chain triglyceride (MCT) oil.

12. The pigment dispersion of claim 1, wherein the liquid oil present in the pigment dispersion is about 25% to 65%, 30% to 60%, 35% to 55%, 40% to 50%, or 45% w/w.

13. The pigment dispersion of claim 1, wherein:
(a) the encapsulated pigment is encapsulated in a water-insoluble protein matrix via extrusion encapsulation;
(b) the protein matrix comprises or consists of extruded plant protein, extruded animal protein, or a mixture thereof; or
(c) the protein matrix comprises or consists of extruded rice protein, extruded pea protein, extruded soy protein, extruded whey protein, extruded sunflower protein, extruded oat protein, extruded fibrous plant protein, or any combination thereof.

14. The pigment dispersion of claim 1, wherein the pigment dispersion comprises or consists of at least two encapsulated pigments of different hues and/or opacities.

15. The pigment dispersion of claim 1, wherein the encapsulated pigment present in the pigment dispersion is between 10% to 60%, 10% to 55%, 15% to 55%, 20% to 55%, 20% to 50%, 25% to 50%, 30% to 50%, 30% to 45%, 35% to 45%, or about 40% w/w; or wherein the encapsulated pigment present in the pigment dispersion is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60% w/w.

16. The pigment dispersion of claim 1, wherein said increase in the stability of the pigment dispersion comprises decreased sedimentation, decreased creaming, decreased clarification, or any combination thereof.

17. A process for preparing a pigment dispersion, the process comprising mixing a solid fat, a liquid oil, and an encapsulated pigment in particulate form, the pigment being encapsulated in a water-insoluble protein matrix, to form a sol colloid having a stability greater than that of a corresponding sol colloid lacking the encapsulated pigment.

18. The process of claim 17, wherein:
(a) at least the solid fat and the liquid oil are mixed under conditions of hydraulic shear sufficiently high to produce a sol colloid, wherein the temperature during sol colloid formation does not exceed the slip melting temperature of the solid fat;
(b) the solid fat and the liquid oil are mixed under conditions of hydraulic shear sufficiently high to produce a sol colloid, into which the encapsulated pigment in particulate form is then dispersed under conditions of lower hydraulic shear;
(c) at least the solid fat and the liquid oil are mixed using a colloid mill; or
(d) any combination of (a) to (c).

19. A pigment dispersion produced by the process of claim 17.

20. A process for increasing the stability of a sol colloid formed between a solid fat and a liquid oil, the process comprising homogenously dispersing therein an extruded water-insoluble protein matrix in particulate form in sufficient quantity to increase the stability of the sol colloid as compared to in the absence of the extruded water-insoluble protein matrix, wherein the extruded water-insoluble protein matrix encapsulates an active agent.
